(19) United States
(12) Patent Application Publication
(10) Pub. No.: US 2022/0304627 A1
(43) Pub. Date: Sep. 29, 2022

(54) BIOLOGICAL INFORMATION MEASUREMENT SYSTEM

(71) Applicants: Hirofumi MORISE, Kanagawa (JP); Kiwamu KUDO, Ishikawa (JP); Yoshihiro MISAKA, Ishikawa (JP)

(72) Inventors: Hirofumi MORISE, Kanagawa (JP); Kiwamu KUDO, Ishikawa (JP); Yoshihiro MISAKA, Ishikawa (JP)

(21) Appl. No.: 17/700,484

(22) Filed: Mar. 22, 2022

(30) Foreign Application Priority Data
Mar. 23, 2021 (JP) 2021-048813

(51) Int. Cl. A61B 5/00; G06T 7/70; G06T 7/20; A61B 5/377

(52) U.S. Cl. CPC A61B 5/721; G06T 7/70; G06T 7/20; A61B 5/377; G06T 2207/30201

(57) ABSTRACT

A biological information measurement system includes: a time measurement apparatus configured to transmit time information; a first measurement apparatus configured to measure brain neural activity of a subject, based on a biological signal detected from the subject; a first recording apparatus configured to record first data indicating a temporal change in the brain neural activity measured by the first measurement apparatus, in association with the time information received from the time measurement apparatus; an image capturing apparatus configured to capture an image of the subject; and a second recording apparatus configured to record second data indicating a temporal change in a posture of the subject, the posture identified based on the image captured by the image capturing apparatus, in association with the time information received from the time measurement apparatus.

### CROSS-REFERENCE TO RELATED APPLICATIONS

The present application claims priority under 35 U.S.C. § 119 to Japanese Patent Application No. 2021-048813, filed on Mar. 23, 2021. The contents of which are incorporated herein by reference in their entirety.

### BACKGROUND OF THE INVENTION

### 1. Field of the Invention

The present invention relates to a biological information measurement system.

### 2. Description of the Related Art

Conventionally, a brain function measurement apparatus, such as a magnetoencephalogram, measures and analyzes a weak biomagnetic field or the like that is generated with human brain neural activity. For example, in a magnetoencephalogram that measures magnetoencephalograph (MEG), a large number of magnetism detectors that are arranged around a head of a subject (or a person subjected to examination) detect a magnetic signal that is generated inside the head. In the brain function measurement apparatus as described above, it is important to determine a positional relationship among sensors, such as the magnetism detectors, and the head. Therefore, the subject is requested not to move a position of the head during measurement, which is a burden on the subject.

Meanwhile, a technology for measuring movement of a head position of a subject while the subject is being subjected to measurement and making synchronization with a measurement result to identify a positional relationship between a sensor and the head of the subject is known. Conventionally, a technology for attaching a contact-type detector capable of detecting a position of a head of a subject onto the head of the subject, and detecting the position of the head of the subject at the time of measurement of a brain function has been proposed (for example, see Japanese Patent No. 6102526). Further, a technology for receiving chronological measurement result data indicating a measurement result on a brain function of a subject measured by a brain function measurement apparatus and a camera video of the subject during the measurement, and generating and recording single screen video data in which a measurement timing of the measurement result and the camera video of the subject at the measurement timing are synchronized has been proposed (for example, see U.S. Pat. No. 7,062,391).

However, in the method of using a contact-type detector, it is common to measure a biomagnetic signal for a long time, and therefore, wearing the detector for a long time may be a burden on a certain subject, such as a child. Further, in the method of using a non-contact type position measuring means, such as an optical camera, it is difficult to synchronize a measurement result of a brain function of a subject and measurement of a head position, so that it may be difficult to obtain an accurate measurement result.

### SUMMARY OF THE INVENTION

A biological information measurement system according to an aspect of the present invention includes a time measurement apparatus, a first measurement apparatus, a first recording apparatus, an image capturing apparatus, and a second recording apparatus. The time measurement apparatus is configured to transmit time information. The first measurement apparatus is configured to measure brain neural activity of a subject, based on a biological signal detected from the subject. The first recording apparatus is configured to record first data indicating a temporal change in the brain neural activity measured by the first measurement apparatus, in association with the time information received from the time measurement apparatus. The image capturing apparatus is configured to capture an image of the subject. The second recording apparatus is configured to record second data indicating a temporal change in a posture of the subject, the posture identified based on the image captured by the image capturing apparatus, in association with the time information received from the time measurement apparatus.

### BRIEF DESCRIPTION OF THE DRAWINGS

FIG. 1 is a diagram illustrating an example of a system configuration of a biological information measurement system according to a first embodiment;

FIG. 2 is a diagram illustrating an example of a hardware configuration of a first information processing apparatus according to the first embodiment;

FIG. 3 is a diagram illustrating an example of a hardware configuration of a second information processing apparatus according to the first embodiment;

FIG. 4 is a diagram illustrating an example of functional configurations of the first information processing apparatus and the second information processing apparatus according to the first embodiment;

FIG. 5 is a sequence diagram illustrating an example of operation performed by a time management apparatus, the first information processing apparatus, and the second information processing apparatus according to the first embodiment;

FIG. 6 is a diagram illustrating an example of functional configurations of a first information processing apparatus and a second information processing apparatus according to a first modification of the first embodiment;

FIG. 7 is a sequence diagram illustrating an example of operation performed by a time management apparatus, the first information processing apparatus, and the second information processing apparatus according to the first modification of the first embodiment;

FIG. 8 is a diagram illustrating an example of a system configuration of a biological information measurement system according to a second embodiment;

FIG. 9 is a diagram illustrating an example of a hardware configuration of a third information processing apparatus according to the second embodiment;

FIG. 10 is a diagram illustrating an example of a functional configuration of the third information processing apparatus according to the second embodiment;

FIG. 11 is a sequence diagram illustrating an example of operation performed by a time management apparatus, the first information processing apparatus, the second information processing apparatus, and the third information processing apparatus according to the second embodiment; and

FIG. 12 is a diagram illustrating an example of a temporal change in a head position according to a third modification of the second embodiment.

The accompanying drawings are intended to depict exemplary embodiments of the present invention and should not be interpreted to limit the scope thereof. Identical or similar reference numerals designate identical or similar components throughout the various drawings.

### DESCRIPTION OF THE EMBODIMENTS

The terminology used herein is for the purpose of describing particular embodiments only and is not intended to be limiting of the present invention.

As used herein, the singular forms “a”, “an” and “the” are intended to include the plural forms as well, unless the context clearly indicates otherwise.

In describing preferred embodiments illustrated in the drawings, specific terminology may be employed for the sake of clarity. However, the disclosure of this patent specification is not intended to be limited to the specific terminology so selected, and it is to be understood that each specific element includes all technical equivalents that have the same function, operate in a similar manner, and achieve a similar result.

An embodiment of the present invention will be described in detail below with reference to the drawings.

An embodiment has an object to measure a brain function with high accuracy without imposing a burden on a subject.

Embodiments of a biological information measurement system will be described in detail below with reference to the accompanying drawings.

### First Embodiment

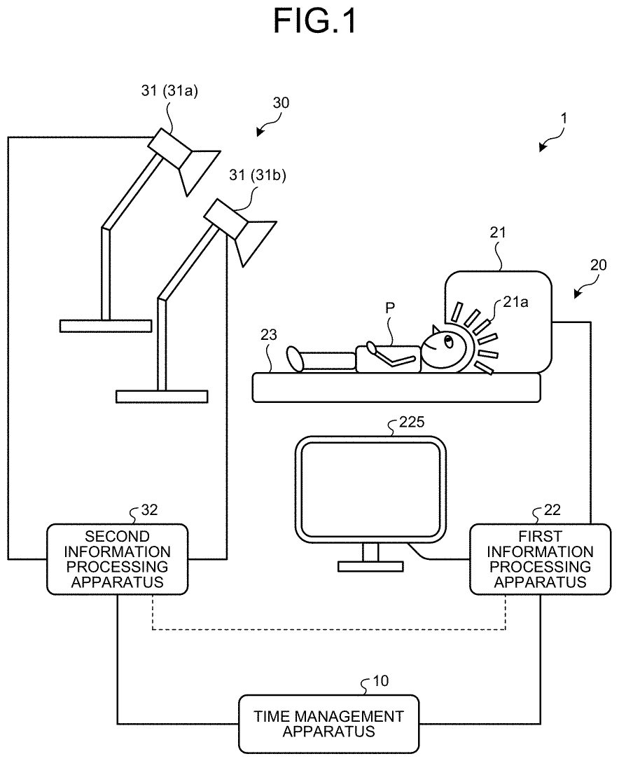

FIG. 1 is a diagram illustrating an example of a system configuration of a biological information measurement system 1 according to a first embodiment. As illustrated in FIG. 1, the biological information measurement system 1 includes a time management apparatus 10, a brain function measurement apparatus 20, and a head position measurement apparatus 30.

The time management apparatus 10 is one example of a time measurement apparatus. The time management apparatus 10 has a time measurement function, such as a real-time clock, to measure a time and transmits time information indicating a measured time to the brain function measurement apparatus 20 (a first information processing apparatus 22 to be described later) and the head position measurement apparatus 30 (a second information processing apparatus 32 to be described later). The way to transmit the time information is not specifically limited, and the time management apparatus 10 may sequentially transmit the time information at a predetermined time interval, such as one second, or may continuously transmit the time information in the form of stream data or the like.

Meanwhile, the time measured by the time management apparatus 10 may be an absolute time, such as Japan Standard Time, or may be unique time in the biological information measurement system 1. Further, the time management apparatus 10 may measure an elapsed time since a certain time that is used as a reference (hereinafter, also referred to as a reference time), for example.

The brain function measurement apparatus 20 is an apparatus or a system for measuring a brain function of a subject P. The brain function measurement apparatus 20 includes a brain function measurement unit 21, the first information processing apparatus 22, and a support table 23.

The brain function measurement unit 21 is one example of a first measurement apparatus. The brain function measurement unit 21 measures brain neural activity of the subject P on the basis of a biological signal detected from the subject P. As one example, the brain function measurement unit 21 is a magnetoencephalogram, an electroencephalogram, or the like, and detects and measures an electrophysiological and biological signal. For example, as illustrated in FIG. 1, the brain function measurement unit 21 has a helmet shape that surrounds almost the entire region of a head of the subject P. A single or a plurality of sensors 21a that are able to detect (measure) a temporal change in an electromagnetic field that is generated on an outer part of the head or a surface of the head and that is caused by the brain neural activity are arranged inside the brain function measurement unit 21. As the sensors 21a, for example, magnetic sensors, potential sensors, or the like may be used. The subject P as a measurement target inserts his/her head into the brain function measurement unit 21. The brain function measurement unit 21 causes the sensors 21a to measure the brain neural activity (brain function) of the subject P, and outputs brain function data that is a measurement result to the first information processing apparatus 22. Here, the brain function data is a single or a plurality of pieces of chronological data that indicate a temporal change in a data item of the measurement target. The brain function data is one example of first data.

If magnetic sensors are used as the sensors 21a, it is preferable to coat a cable or the like that connects image capturing apparatuses 31 (to be described later) and various devices with a soft magnetic material, such as a soft magnetic film, to prevent an influence of magnetic noise that occurs from a camera or the cable.

The first information processing apparatus 22 is one example of a first recording apparatus. The first information processing apparatus 22 is implemented by, for example, an information processing apparatus, such as a personal computer (PC). The first information processing apparatus 22 is connected to the time management apparatus 10 in a wired or wireless manner, and sequentially receives the time information transmitted from the time management apparatus 10. Further, the first information processing apparatus 22 is connected to the brain function measurement unit 21 in a wired or wireless manner, and sequentially receives the brain function data measured by the brain function measurement unit 21. Then, the first information processing apparatus 22 records (or stores) the received time information and the received brain function data in a storage unit 224 (to be described later) in an associated manner.

The support table 23 is, for example, a bed, and supports the subject P being subjected to measurement. In FIG. 1, the support table 23 supports the subject P in a supine position, but may be configured to support the subject P in a different body position, such as a standing position or a sitting position.

Meanwhile, in the present embodiment, the brain function measurement apparatus 20 includes the support table 23, but the brain function measurement apparatus 20 may be configured without the support table 23. Further, the first information processing apparatus 22 may be connected to the second information processing apparatus 32 (to be described later) in a wired or wireless manner.

The head position measurement apparatus 30 is an apparatus or a system for measuring a head position of the subject P. The head position measurement apparatus 30 includes the image capturing apparatuses 31 and the second information processing apparatus 32.

The image capturing apparatuses 31 are one example of an image capturing apparatus. The image capturing apparatuses 31 are arranged on the outside of the brain function measurement apparatus 20 and capture images of the subject P. Specifically, the image capturing apparatuses 31 capture images of a part of a face or the head of the subject P at a predetermined time interval, and transmit captured images (hereinafter, also referred to as captured image data) obtained by image capturing to the second information processing apparatus 32. The image capturing apparatuses 31 may sequentially transmit the captured image data every time the image capturing apparatuses 31 perform image capturing, or may temporarily store the captured image data in a memory region (not illustrated) and transmit a plurality of pieces of captured image data in chronological order.

FIG. 1 illustrates an example in which the two image capturing apparatuses 31, that is, an image capturing apparatus 31a and an image capturing apparatus 31b, are provided, but embodiments are not limited to this example, and the number of the image capturing apparatuses may be one or three or more. Further, each of the image capturing apparatuses 31 may include a single or a plurality of cameras. Furthermore, the number of image capturing units (for example, a lens and an imaging element) included in each of the cameras is not limited to one, but may be plural. In this case, the image capturing apparatuses 31 function as depth camera systems, acquire captured image data including a distance to the subject P, and transmit the captured image data to the second information processing apparatus 32.

Moreover, when the image capturing apparatuses 31 capture images, it is preferable to capture images at an image capturing interval (image capturing frequency) that is the same as a frequency of commercial power for supplying power to the biological information measurement system 1 (or the image capturing apparatuses 31) or that is a multiple of the frequency. With this configuration, by performing a bandpass filtering process, it is possible to remove noise that is caused by image capturing operation of the image capturing apparatuses 31 in addition to noise that is caused by commercial power, so that it is possible to reduce an influence of the noise on the brain function data. The bandpass filtering process may be performed by the image capturing apparatuses 31 or the second information processing apparatus 32.

The second information processing apparatus 32 is one example of a second recording apparatus. The second information processing apparatus 32 is implemented by, for example, a certain information processing apparatus, such as a PC. The second information processing apparatus 32 is connected to the time management apparatus 10 in a wired or wireless manner, and sequentially receives the time information transmitted from the time management apparatus 10. Further, the second information processing apparatus 32 is connected to the image capturing apparatuses 31 in a wired or wireless manner, and sequentially receives the captured image data obtained by the image capturing apparatus 31.

Furthermore, the second information processing apparatus 32 estimates a posture of the subject P from the captured image data obtained by the image capturing apparatuses 31. Specifically, the second information processing apparatus 32 estimates a position, an orientation, a movement amount (hereinafter, also collectively referred to as a head position) of the face (hereinafter, also referred to as the head) of the subject P. Then, the second information processing apparatus 32 records (or stores) the received time information and head position data indicating a temporal change in the estimated head position in a storage unit 324 (to be described later) in an associated manner. The head position data is one example of second data.

Configurations of the first information processing apparatus 22 and the second information processing apparatus 32 as described above will be described below.

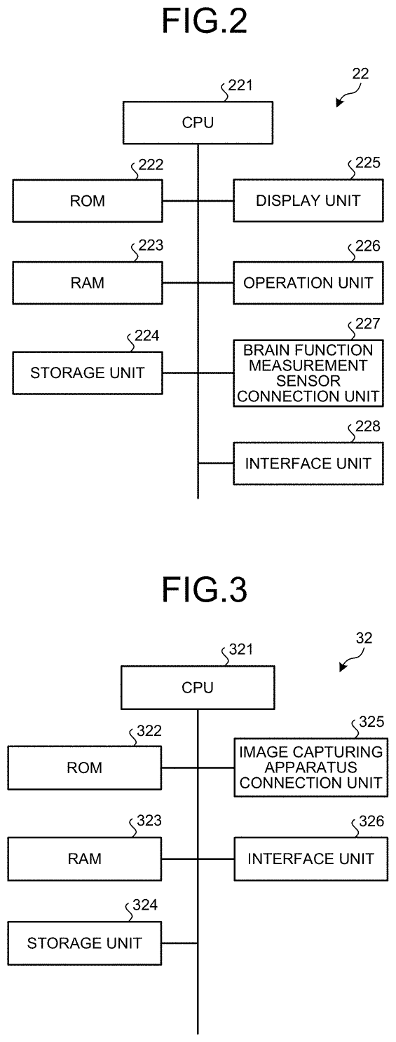

FIG. 2 is a diagram illustrating an example of a hardware configuration of the first information processing apparatus 22. As illustrated in FIG. 2, the first information processing apparatus 22 includes a central processing unit (CPU) 221, a read only memory (ROM) 222, a random access memory (RAM) 223, the storage unit 224, a display unit 225, an operation unit 226, a brain function measurement sensor connection unit 227, and an interface unit 228.

The CPU 221 is one example of a processor and integrally controls each of the units of the first information processing apparatus 22. The ROM 222 stores therein various programs. The RAM 223 is a workspace for loading a program and various kinds of data. The CPU 221, the ROM 222, and the RAM 223 implement a computer configuration of the first information processing apparatus 22, and function as a control unit of the first information processing apparatus 22.

The storage unit 224 is a storage device, such as a hard disk drive (HDD) or a flash memory. The storage unit 224 stores therein various programs executed by the CPU 221, setting information, and the like. Further, the storage unit 224 functions as a storage area for storing the brain function data.

The display unit 225 is a display, such as a liquid crystal display (LCD). The display unit 225 displays various kinds of information and screens under the control of the CPU 221. The operation unit 226 includes an input device, such as a keyboard or a mouse, and outputs a signal corresponding to user operation to the CPU 221. The operation unit 226 may be a touch panel that is arranged on a surface of the display unit 225.

The brain function measurement sensor connection unit 227 is an interface for connecting the brain function measurement unit 21. The interface unit 228 is an interface for performing communication with the time management apparatus 10. The interface unit 223 may perform communication with an external apparatus, such as the second information processing apparatus 32.

FIG. 3 is a diagram illustrating an example of a hardware configuration of the second information processing apparatus 32. As illustrated in FIG. 3, the second information processing apparatus 32 includes a CPU 321, a ROM 322, a RAM 323, the storage unit 324, an image capturing apparatus connection unit 325, and an interface unit 326.

The CPU 321 is one example of a processor and integrally controls each of the units of the second information processing apparatus 32. The ROM 322 stores therein various programs. The RAM 323 is a workspace for loading a program and various kinds of data. The CPU 321, the ROM 322, and the RAM 323 implement a computer configuration of the second information processing apparatus 32, and function as a control unit of the second information processing apparatus 32.

The storage unit 324 is a storage device, such as a HDD or a flash memory. The storage unit 324 stores therein various programs executed by the CPU 321, setting information, and the like. Further, the storage unit 324 functions as a storage area for storing the head position data.

The image capturing apparatus connection unit 325 is an interface for connecting the image capturing apparatus 31. The interface unit 326 is an interface for performing communication with the time management apparatus 10. The interface unit 326 may perform communication with an external apparatus, such as the first information processing apparatus 22.

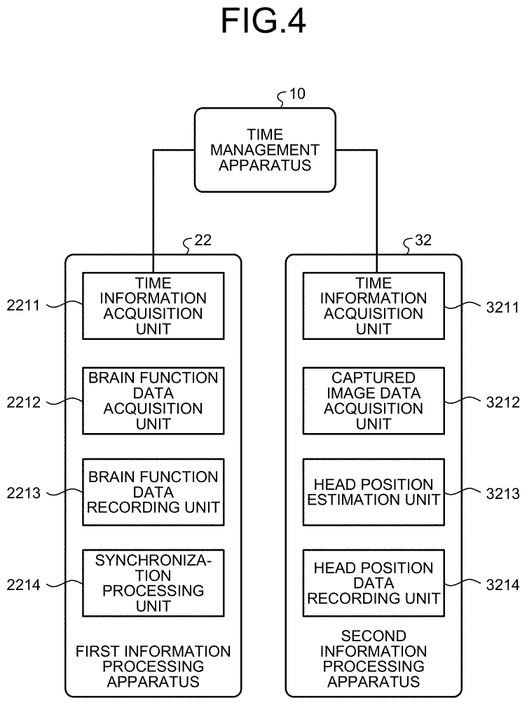

Functional configurations of the first information processing apparatus 22 and the second information processing apparatus 32 will be described below. FIG. 4 is a diagram illustrating an example of the functional configurations of the first information processing apparatus 22 and the second information processing apparatus 32. In FIG. 4, the time management apparatus 10 is illustrated for convenience of explanation.

As illustrated in FIG. 4, the first information processing apparatus 22 includes, as functional units, a time information acquisition unit 2211, a brain function data acquisition unit 2212, a brain function data recording unit 2213, and a synchronization processing unit 2214.

A part or all of the functional units included in the first information processing apparatus 22 may have a software configuration that is implemented by cooperation of a processor (for example, the CPU 221) of the first information processing apparatus 22 and a program stored in a memory (for example, the ROM 222 or the storage unit 224). Further, a part or all of the functional units included in the first information processing apparatus 22 may have a hardware configuration implemented by a dedicated circuit or the like that is mounted on the first information processing apparatus 22.

The time information acquisition unit 2211 acquires (or receives) the time information transmitted from the time management apparatus 10, in cooperation with the interface unit 228. Specifically, the time information acquisition unit 2211 sequentially acquires the time information transmitted from the time management apparatus 10.

The brain function data acquisition unit 2212 acquires (or receives) the brain function data transmitted from the brain function measurement unit 21, in cooperation with the brain function measurement sensor connection unit 227. Specifically, the brain function data acquisition unit 2212 sequentially acquires the brain function data transmitted from the brain function measurement unit 21.

Meanwhile, the brain function data acquisition unit 2212 may perform predetermined signal processing on the acquired brain function data. For example, the brain function data acquisition unit 2212 may perform signal processing, such as noise removal or bandpass filtering, on the acquired brain function data.

The brain function data recording unit 2213 sequentially records (or stores) the brain function data acquired by the brain function data acquisition unit 2212 in the storage unit 224 in association with the time information acquired by the time information acquisition unit 2211.

Meanwhile, the brain function data recording unit 2213 may record the brain function data and the time information as integrated data or may record the brain function data and the time information as separate data. In the former case, for example, the brain function data recording unit 2213 may record a time indicated by the time information as a timeline in an integrated manner with the brain function data.

The synchronization processing unit 2214 is a functional unit that performs a process of synchronizing the brain function data that is recorded by the subject apparatus and the head position data that is recorded by the second information processing apparatus 32, on the basis of the time information that is recorded together with the above-described data. The synchronization processing unit 2214 will be described later.

In contrast, the second information processing apparatus 32 includes, as functional units, a time information acquisition unit 3211, a captured image data acquisition unit 3212, a head position estimation unit 3213, and a head position data recording unit 3214.

A part or all of the functional units included in the second information processing apparatus 32 may have a software configuration that is implemented by cooperation of a processor (for example, the CPU 321) of the second information processing apparatus 32 and a program stored in a memory (for example, the ROM 322 or the storage unit 324). Further, a part or all of the functional units included in the second information processing apparatus 32 may have a hardware configuration implemented by a dedicated circuit or the like that is mounted on the second information processing apparatus 32.

The time information acquisition unit 3211 acquires (or receives) the time information transmitted from the time management apparatus 10, in cooperation with the interface unit 326. Specifically, the time information acquisition unit 3211 sequentially acquires the time information transmitted from the time management apparatus 10.

The captured image data acquisition unit 3212 acquires (or receives) the captured image data transmitted from the image capturing apparatus 31, in cooperation with the image capturing apparatus connection unit 325. Specifically, the captured image data acquisition unit 3212 sequentially acquires the captured image data transmitted from the image capturing apparatus 31.

The head position estimation unit 3213 estimates the head position indicating the position, the orientation, or the like of the head of the subject P, on the basis of the captured image data acquired by the captured image data acquisition unit 3212. Further, the head position estimation unit 3213 sequentially estimates the head position of the subject P, and generates chronological data (hereinafter, also referred to as head position data) indicating a chronological change in the head position. Furthermore, a method of estimating the head position is not specifically limited, and various methods may be adopted.

For example, the head position estimation unit 3213 may detect a feature of the face of the subject P from the captured image data, and estimate the head position from the detected feature. In this case, it may be possible to detect a feature, such as a relative arrangement position or a shape of eyes, a nose, a mouth, or the like of the subject P, as the feature of the face.

Furthermore, for example, the head position estimation unit 3213 may estimate the head position of the subject P by using a feature obtained from the captured image data and a trained model that is obtained by performing machine learning on a relationship with the head position. In this case, the head position estimation unit 3213 inputs the captured image data acquired by the captured image data acquisition unit 3212 into the trained model, and acquires an estimation result output by the trained model as the head position of the subject P.

Moreover, if the image capturing apparatuses 31 function as depth camera systems, the head position estimation unit 3213 may acquire a three-dimensional shape of the face from the captured image data, compare the three-dimensional shape with structural data that is acquired in advance and that indicates a three-dimensional shape of the face of the subject P, and estimate the orientation and the position of the head. In this case, the structural data of the face of the subject P may be acquired by using a modality, such as magnetic resonance imaging (MRI).

The head position data recording unit 3214 sequentially records (or stores) the head position data generated by the head position estimation unit 3213 in the storage unit 324 in association with the time information acquired by the time information acquisition unit 3211.

Meanwhile, the head position data recording unit 3214 may record the head position data and the time information in an integrated manner or may record the head position data and the time information as separate data. In the former case, for example, the head position data recording unit 3214 may record a time indicated by the time information as a timeline in an integrated manner with the head position data.

The synchronization processing unit 2214 as described above acquires the head position data and the time information that are recorded by the second information processing apparatus 32, by online or offline. Then, the synchronization processing unit 2214 performs a process of synchronizing the brain function data and the head position data on the basis of the acquired time information on the head position data and the time information on the head position data recorded in the subject apparatus. For example, the synchronization processing unit 2214 may generate data in which the brain function data and the head position data are synchronized in accordance with the time information that is used in common. Further, the synchronization processing unit 2214 may output the brain function data and head position data that are synchronized with each other to the display unit 225 or the like.

Meanwhile, in the present embodiment, synchronization is not limited to an exact temporal match, but may include temporal deviation. For example, synchronization may include temporal deviation up to one second.

Furthermore, in the present embodiment, the first information processing apparatus 22 is configured to include the synchronization processing unit 2214, but embodiments are not limited to this example, and a different apparatus, such as the second information processing apparatus 32, may include the synchronization processing unit 2214. In any case, the synchronization processing unit 2214 performs the process of synchronizing the brain function data and the head position data that are recorded by the first information processing apparatus 22 and the second information processing apparatus 32, on the basis of the time information that is recorded in association with each of the brain function data and the head position data.

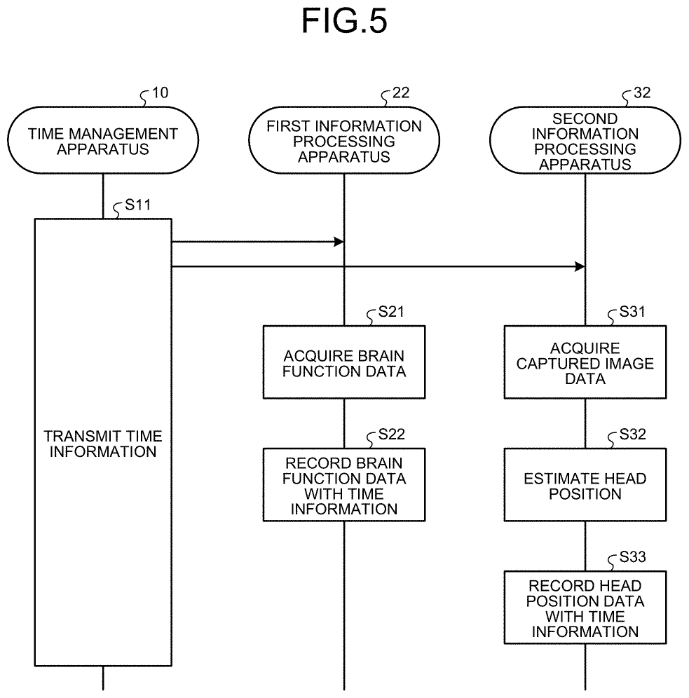

An example of operation performed by the biological information measurement system 1 according to the present embodiment will be described below. FIG. 5 is a sequence diagram illustrating an example of operation performed by the time management apparatus 10, the first information processing apparatus 22, and the second information processing apparatus 32.

First, the time management apparatus 10 transmits the time information to the first information processing apparatus 22 and the second information processing apparatus 32 (Step S11). Here, the time management apparatus 10 transmits the time information at a predetermined time interval or in a continuous manner so as to sequentially transmit the time information while the brain function of the subject P is being measured. Further, with this operation, the time information acquisition unit 2211 of the first information processing apparatus 22 and the time information acquisition unit 3211 of the second information processing apparatus 32 sequentially receive the time information transmitted from the time management apparatus 10.

Under the condition as described above, the first information processing apparatus 22 performs a process of recording the brain function data measured by the brain function measurement unit 21. Specifically, if the brain function data acquisition unit 2212 acquires the brain function data (Step S21), the brain function data recording unit 2213 records the acquired brain function data in the storage unit 224 in association with the time information (Step S22).

Further, the second information processing apparatus 32 performs a process of recording the head position of the subject P on the basis of the captured image data obtained by the image capturing apparatus 31. Specifically, if the captured image data acquisition unit 3212 acquires the captured image data (Step S31), the head position estimation unit 3213 estimates the head position of the subject P on the basis of the acquired captured image data (Step S32). Then, the head position data recording unit 3214 records the head position data that indicates the estimated head position in the storage unit 324, in association with the time information (Step S33).

As described above, the first information processing apparatus 22 of the present embodiment records the time information transmitted from the time management apparatus 10 in association with the brain function data. Further, the second information processing apparatus 32 records the time information transmitted from the time management apparatus 10 in association with the head position data. With this configuration, in the biological information measurement system 1, each of the brain function measurement unit 21 and the second information processing apparatus 32 is able to synchronize the brain function data and the head position data on the basis of the time information that is recorded together with the brain function data and the head position data. Therefore, in the biological information measurement system 1, it is possible to obtain the brain function data and the head position data that can be temporally synchronized with each other without imposing a burden on the subject P, so that it is possible to measure the brain function with high accuracy.

In general, the brain function data enables a process corresponding to an amount of deviation of the head position or correction corresponding to the amount of deviation of the head position, by being combined with the head position data synchronized with the brain function data. Therefore, for example, in various processes, such as examinations related to psychiatric or neurological disorder or the like or feature detection of brain neural activity in a resting state, in a sleeping state, with an evoked response, in a certain psychological state, or the like, it is possible to obtain data with high accuracy.

Meanwhile, in the embodiment as described above, a timing at which the brain function measurement apparatus 20 measures the brain function and a timing at which the head position measurement apparatus 30 measures the head position are not specifically described; however, the measurement may be performed at the same timing or at independent timings. For example, if image capturing operation performed by the image capturing apparatus 31 causes noise for measurement of the brain function, it may be possible to perform image capturing before or after the measurement of the brain function, and the head position data during the measurement of the brain function may be estimated from the captured image data obtained by image capturing.

Furthermore, an electrophysiological brain function measurement apparatus, such as a magnetoencephalogram or an electroencephalogram, measures a change in the brain neural activity with high time resolution, such as 0.1 millisecond to 1000 millisecond, but movement of the head of the subject P occurs with a slower time scale. Therefore, even if the image capturing apparatus 31 capture images during the measurement of the brain function, it is sufficient that an image capturing frequency is lower than a frequency of the measurement of the brain function. For example, by setting the image capturing frequency to 20 milliseconds or more, it is possible to obtain data with high accuracy while reducing an influence of the noise.

Meanwhile, in this case, the head position (head position data) associated with the brain function data may be a representative value (for example, an average value, a mean value, or the like) in a predetermined time period, or may be a value based on a regression equation, such as a linear regression equation. Further, with use of the representative value or the value based on a regression equation, it is possible to reduce an influence of an error that is caused by estimation of the head position.

Furthermore, the biological information measurement system 1 is not limited to electrophysiological measurement, such as a magnetoencephalogram or an electroencephalogram, but may be adopted for detection of a change in blood flow or metabolism. For example, the biological information measurement system 1 may be adopted for various brain function measurement apparatuses (modalities), such as functional magnetic resonance imaging (fMRI), functional near-infrared spectroscopy (fNIRS), or positron emission tomography (PET). However, the electrophysiological measurement has higher time resolution than fMRI, fNIRS, and the like so that the effect of highly accurate synchronization in the form as described above can largely contribute to the electrophysiological measurement, and therefore the electrophysiological measurement is preferable.

The embodiment as described above may be embodied with various modifications by changing a part of the configurations and functions of each of the apparatuses as described above. Therefore, in the following, some modifications of the embodiment as described above will be described as other examples. In the following, a difference from the embodiment as described above will be mainly described and detailed explanation of components common to those as described above will be omitted. Further, the modifications described below may be implemented individually or may be implemented by an appropriate combination.

First Modification

In the embodiment as described above, the mode in which the time management apparatus 10 continuously transmits the time information has been described. However, if the first information processing apparatus 22 and the second information processing apparatus 32 have time measurement functions, such as real-time clocks (RTC), each of the first information processing apparatus 22 and the second information processing apparatus 32 is able to synchronize the brain function data and the head position data by measuring time based on the time information transmitted from the time management apparatus 10. A configuration example of the present modification will be described below.

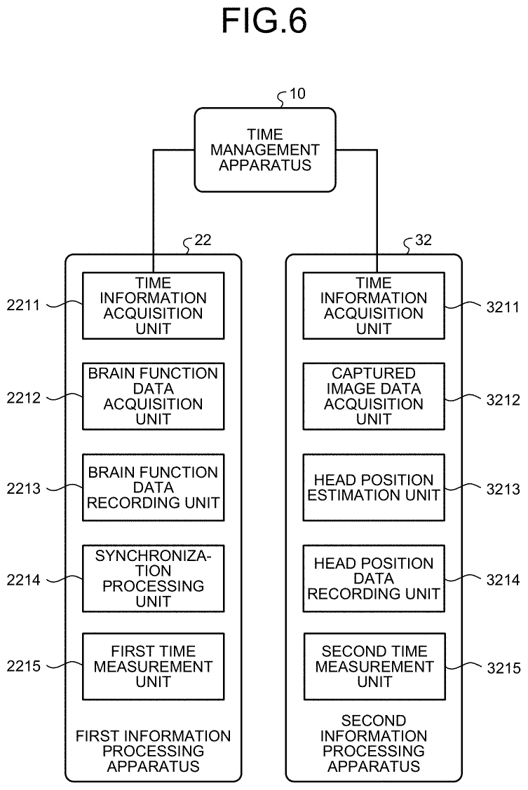

FIG. 6 is a diagram illustrating an example of functional configurations of the first information processing apparatus 22 and the second information processing apparatus 32 according to the present modification. In FIG. 6, the time management apparatus 10 is illustrated similarly to FIG. 4.

As illustrated in FIG. 6, the first information processing apparatus 22 includes, as functional units, a first time measurement unit 2215 in addition to the time information acquisition unit 2211, the brain function data acquisition unit 2212, the brain function data recording unit 2213, and the synchronization processing unit 2214 as described above.

The first time measurement unit 2215 is one example of a first time measurement unit. The first time measurement unit 2215 measures a time in cooperation with a time measurement apparatus, such as an RTC, that is included in the first information processing apparatus 22. Specifically, if the time information acquisition unit 2211 acquires the time information, the first time measurement unit 2215 starts to measure a time in synchronization with the time information.

The second information processing apparatus 32 includes, as functional units, a second time measurement unit 3215 in addition to the time information acquisition unit 3211, the captured image data acquisition unit 3212, the head position estimation unit 3213, and the head position data recording unit 3214 as described above.

The second time measurement unit 3215 is one example of a second time measurement unit. The second time measurement unit 3215 measures a time in cooperation with a time measurement apparatus, such as an RTC, that is included in the second information processing apparatus 32. Specifically, if the time information acquisition unit 3211 acquires the time information, the second time measurement unit 3215 starts to measure a time in synchronization with the time information.

With the functional units such as the first time measurement unit 2215 and the second time measurement unit 3215 as described above, each of the first information processing apparatus 22 and the second information processing apparatus 32 generates time information that is synchronized with a time measured by the time management apparatus 10.

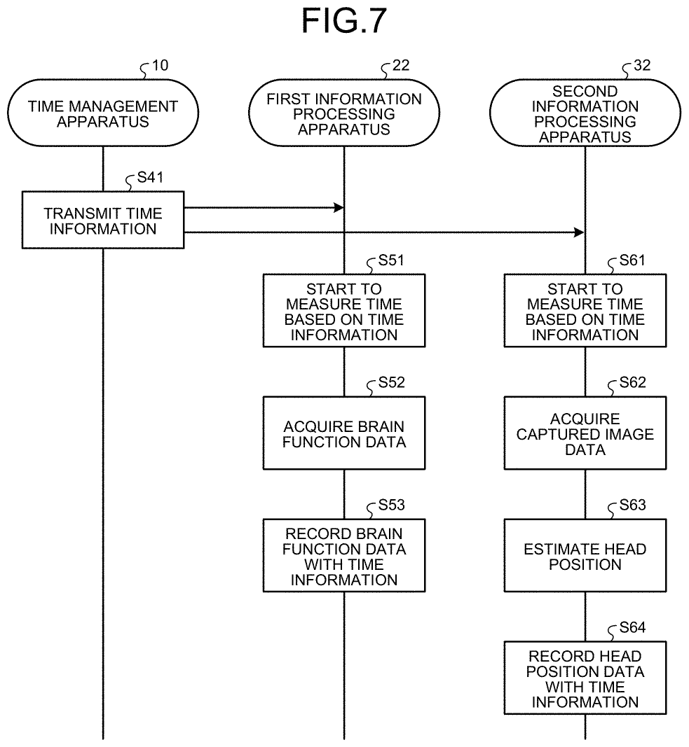

An example of operation performed by the biological information measurement system 1 according to the present modification will be described below. FIG. 7 is a sequence diagram illustrating an example of operation performed by the time management apparatus 10, the first information processing apparatus 22, and the second information processing apparatus 32 according to the present modification.

First, the time management apparatus 10 transmits the time information to the first information processing apparatus 22 and the second information processing apparatus 32 (Step S41). Here, the time management apparatus 10 transmits the time information once or a plurality of number of times.

In the first information processing apparatus 22, if the time information acquisition unit 2211 the time information transmitted from the time management apparatus 10, the first time measurement unit 2215 starts to measure a time based on the time information (Step S51). Subsequently, if the brain function data acquisition unit 2212 acquires the brain function data (Step S52), the brain function data recording unit 2213 records the acquired brain function data in the storage unit 224 in association with the time information measured by the first time measurement unit 2215 (Step S53).

Further, in the second information processing apparatus 32, if the time information acquisition unit 3211 acquires the time information transmitted from the time management apparatus 10, the second time measurement unit 3215 starts to measure a time based on the time information (Step S61). Subsequently, if the captured image data acquisition unit 3212 acquires the captured image data (Step S62), the head position estimation unit 3213 estimates the head position of the subject P on the basis of the acquired captured image data (Step S63). Then, the head position data recording unit 3214 records the head position data indicating the estimated head position in the storage unit 324 in association with the time information measured by the second time measurement unit 3215 (Step S64).

As described above, in the biological information measurement system 1 according to the present modification, each of the first information processing apparatus 22 and the second information processing apparatus 32 measures the time information that is synchronized with the time measured by the time management apparatus 10. Then, each of the first information processing apparatus 22 and the second information processing apparatus 32 records the time information that is individually measured, together with the brain function data or the head position data.

With this configuration, in the configuration according to the present modification, similarly to the embodiment as described above, it is possible to temporally synchronize the brain function data and the head position data on the basis of the time information that is measured by the first information processing apparatus 22 or the second information processing apparatus 32. Therefore, it is possible to achieve the same effects as those of the embodiment as described above.

Second Modification

In the embodiment as described above, the configuration in which the time management apparatus 10 is an independent apparatus has been described, but embodiments are not limited to this example, and a different apparatus, such as the first information processing apparatus 22, the second information processing apparatus 32, or the like may have the functions of the time management apparatus 10.

For example, if the first information processing apparatus 22 has the functions of the time management apparatus 10, the first information processing apparatus 22 transmits the time information that the first information processing apparatus 22 has measured to the second information processing apparatus 32. The first information processing apparatus 22 also records the acquired brain function data, in association with the time information that the first information processing apparatus 22 has measured.

For example, if the second information processing apparatus 32 has the functions of the time management apparatus 10, the second information processing apparatus 32 transmits the time information that the second information processing apparatus 32 has measured to the first information processing apparatus 22. The second information processing apparatus 32 also records the acquired head position data, in association with the time information that the second information processing apparatus 32 has measured.

With this configuration, in the configuration according to the present modification, similarly to the embodiment as described above, it is possible to temporally synchronize the brain function data and the head position data on the basis of the time information that is measured by the first information processing apparatus 22 or the second information processing apparatus 32. Therefore, it is possible to achieve the same effects as those of the embodiment as described above.

### Second Embodiment

In a second embodiment, a configuration in which an apparatus that gives a stimulus to the subject P is included in addition to the components of the biological information measurement system 1 as described above will be described. Meanwhile, the same components as those of the embodiment as described above will be denoted by the same reference symbols and explanation thereof will be omitted.

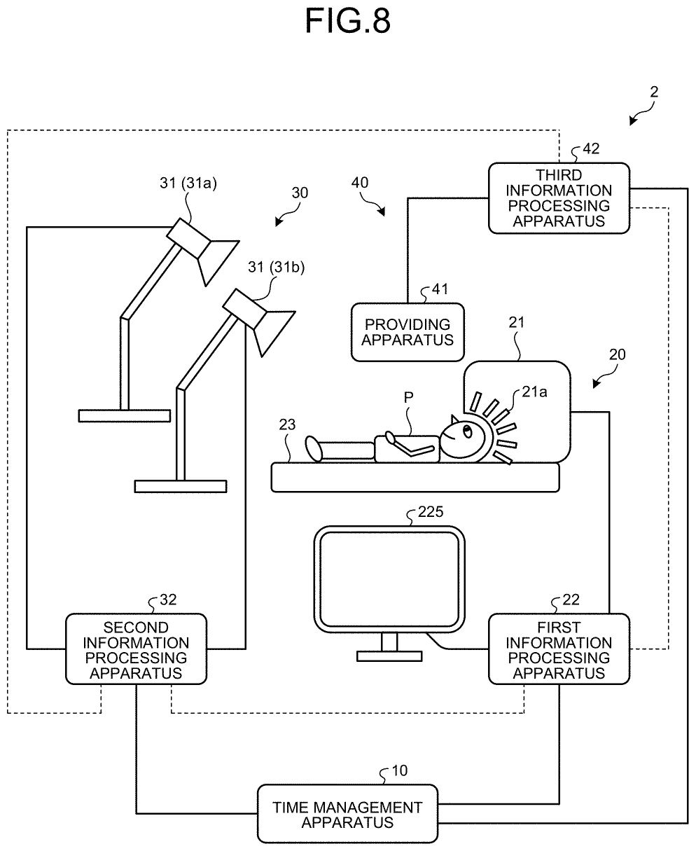

FIG. 8 is a diagram illustrating an example of a system configuration of a biological information measurement system 2 according to the second embodiment. As illustrated in FIG. 8, the biological information measurement system 2 includes the time management apparatus 10, the brain function measurement apparatus 20, the head position measurement apparatus 30, and a stimulus providing apparatus 40.

The stimulus providing apparatus 40 is an apparatus or a system that gives a stimulus to the subject P. The stimulus providing apparatus 40 includes a providing apparatus 41 and a third information processing apparatus 42.

The providing apparatus 41 is one example of a providing apparatus. The providing apparatus 41 is an apparatus that provides a stimulus to the subject P. The providing apparatus 41 is connected to the third information processing apparatus 42, and provides a stimulus to the subject P under the control of the third information processing apparatus 42.

Here, a type of the stimulus to be provided to the subject P is not specifically limited, and it may be possible to provide various stimuli. For example, if an auditory stimulus is to be provided to the subject P, the providing apparatus 41 may be implemented by a speaker, a headphone, or the like that outputs audio. For another example, if a somatosensory stimulus is to be provided to the subject P, the providing apparatus 41 may be implemented by an apparatus configuration that is able to give an electrical stimulus or a mechanical contact stimulus to a leg or a hand of the subject P. For still another example, if an optical stimulus is to be provided to the subject P, the providing apparatus 41 may be implemented by a light source device or the like that emits light.

For still another example, if a visual stimulus is to be provided to the subject P, the providing apparatus 41 may be implemented by a display device, such as a liquid crystal display, a projector and a screen for projecting an output video, or the like. In this case, the display device or the screen is arranged at a position that is included in the field of view of the subject P being subjected to measurement. Further, if the projector is used, it is preferable to use a mirror or the like to prevent an electromagnetic influence on the measurement. Meanwhile, it is assumed that the image capturing apparatuses 31 are arranged at positions at which it is possible to capture images of the head or the face of the subject P.

The third information processing apparatus 42 is one example of a third recording apparatus. The third information processing apparatus 42 is implemented by, for example, an information processing apparatus, such as a PC. The third information processing apparatus 42 is connected to the time management apparatus 10 in a wired or wireless manner, and sequentially receives the time information transmitted from the time management apparatus 10. Further, the third information processing apparatus 42 is connected to the providing apparatus 41 in a wired or wireless manner, and transmits, to the providing apparatus 41, instruction information for giving an instruction on stimulus content to be provided. Furthermore, the third information processing apparatus 42 records (or stores) the received time information and the stimulus content for which the instruction is given to the providing apparatus 41 in a storage unit 424 (to be described later) in an associated manner.

Meanwhile, the third information processing apparatus 42 may be connected to the first information processing apparatus 22 and the second information processing apparatus 32 in a wired or wireless manner.

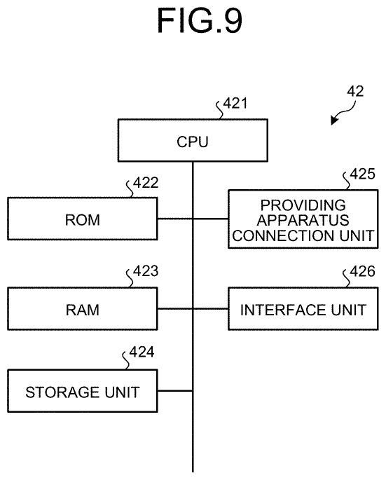

FIG. 9 is a diagram illustrating an example of a hardware configuration of the third information processing apparatus 42. As illustrated in FIG. 9, the third information processing apparatus 42 includes a CPU 421, a ROM 422, a RAM 423, the storage unit 424, a providing apparatus connection unit 425, and an interface unit 426.

The CPU 421 is one example of a processor and integrally controls each of the units of the third information processing apparatus 42. The ROM 422 stores therein various programs. The RAM 423 is a workspace for loading a program and various kinds of data. The CPU 421, the ROM 422, and the RAM 423 implement a computer configuration of the third information processing apparatus 42, and function as a control unit of the third information processing apparatus 42.

The storage unit 424 is a storage device, such as a HDD or a flash memory. The storage unit 424 stores therein various programs executed by the CPU 421, setting information, and the like. Further, the storage unit 424 functions as a storage area for storing the stimulus content provided to the subject P.

The providing apparatus connection unit 425 is an interface for connecting the providing apparatus 41. The interface unit 426 is an interface for performing communication with the time management apparatus 10. Meanwhile, the interface unit 426 may perform communication with an external apparatus, such as the first information processing apparatus 22, the second information processing apparatus 32, or the like.

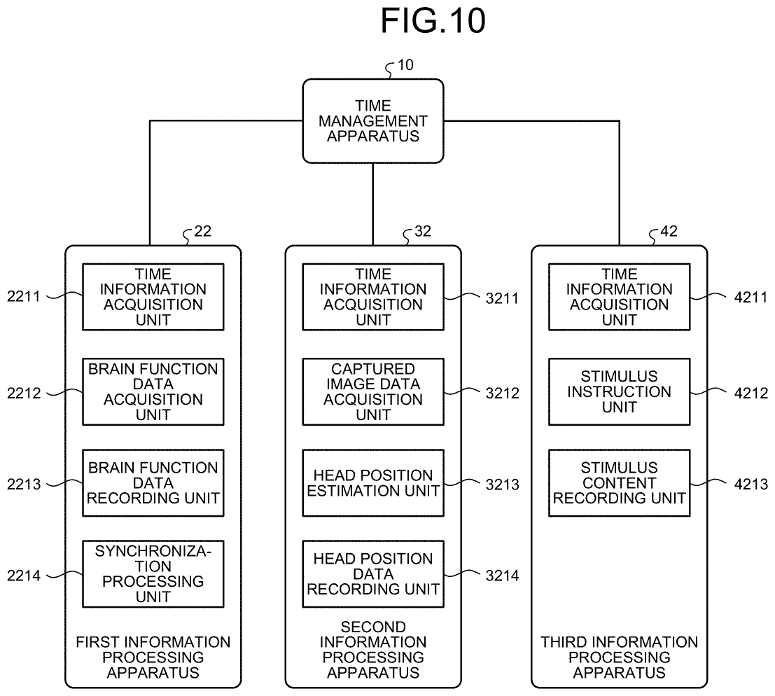

FIG. 10 is a diagram illustrating an example of a functional configuration of the third information processing apparatus 42. In FIG. 10, for convenience of explanation, the time management apparatus 10, the first information processing apparatus 22, and the second information processing apparatus 32 are illustrated together.

As illustrated in FIG. 10, the third information processing apparatus 42 includes, as functional units, a time information acquisition unit 4211, a stimulus instruction unit 4212, and a stimulus content recording unit 4213.

A part or all of the functional units included in the third information processing apparatus 42 may have a software configuration that is implemented by cooperation of a processor (for example, the CPU 421) of the third information processing apparatus 42 and a program stored in a memory (for example, the ROM 422 or the storage unit 424). Further, a part or all of the functional units included in the third information processing apparatus 42 may have a hardware configuration implemented by a dedicated circuit or the like that is mounted on the third information processing apparatus 42.

The time information acquisition unit 4211 acquires (or receives) the time information transmitted from the time management apparatus 10, in cooperation with the interface unit 426. Specifically, the time information acquisition unit 4211 sequentially acquires the time information transmitted from the time management apparatus 10.

The stimulus instruction unit 4212 transmits the instruction information indicating the stimulus content to be provided to the providing apparatus 41 connected to the providing apparatus connection unit 425, and provides a stimulus to the subject P. Here, the stimulus instruction unit 4212 may transmit the instruction information in accordance with user operation, or may transmit the instruction information on the basis of a procedure or setting that is determined in advance.

The stimulus content recording unit 4213 sequentially records (or stores) the stimulus content indicated by the stimulus instruction unit 4212 in the storage unit 424, in association with the time information acquired by the time information acquisition unit 4211.

The stimulus content recording unit 4213 may record the stimulus content and the time information as integrated data or separate data. In the former case, for example, the stimulus content recording unit 4213 may record chronological data indicating a temporal change in the stimulus content by using a time, as a timeline, indicated by the time information.

Meanwhile, the synchronization processing unit 2214 acquires the head position data and the time information that are recorded by the second information processing apparatus 32 and the stimulus content and the time information that are recorded by the third information processing apparatus 42, by online or offline. Then, the synchronization processing unit 2214 performs a process of synchronizing the pieces of data on the basis of the acquired time information on the data and the time information on the head position data recorded in the subject apparatus.

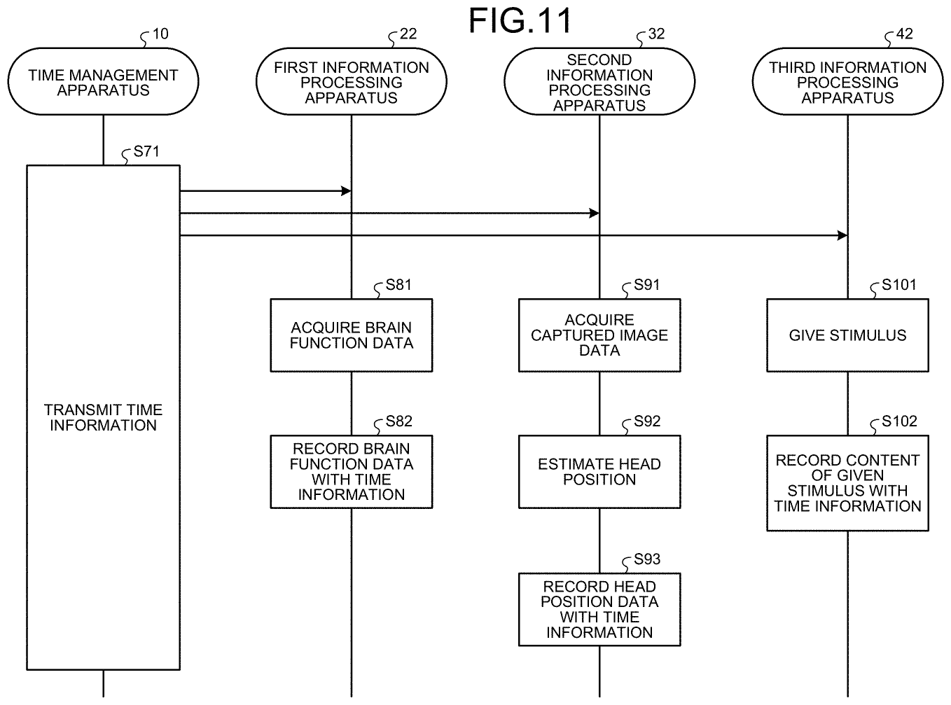

An example of operation performed by the biological information measurement system 2 according to the present embodiment will be described below. FIG. 11 is a sequence diagram illustrating an example of operation performed by the time management apparatus 10, the first information processing apparatus 22, the second information processing apparatus 32, and the third information processing apparatus 42.

First, the time management apparatus 10 sequentially transmits the time information to the first information processing apparatus 22, the second information processing apparatus 32, and the third information processing apparatus 42 (Step S71). Accordingly, the time information acquisition unit 2211 of the first information processing apparatus 22, the time information acquisition unit 3211 the second information processing apparatus 32, and the time information acquisition unit 4211 of the third information processing apparatus 42 sequentially receive the time information transmitted from the time management apparatus 10.

Meanwhile, Steps S81 and S82 performed by the first information processing apparatus 22 and Steps S91 to S93 performed by the second information processing apparatus 32 are the same as Steps S21 and S22 performed by the first information processing apparatus 22 and Steps S31 to S33 performed by the second information processing apparatus 32 illustrated in FIG. 5, and therefore, explanation thereof will be omitted.

Under the condition as described above, the third information processing apparatus 42 performs a process of recording the stimulus content that are provided using the providing apparatus 41. Specifically, the stimulus instruction unit 4212 transmits the instruction information indicating the stimulus content to be provided to the subject P to the providing apparatus 41 and gives a stimulus to the subject P (Step S101). Subsequently, the stimulus content recording unit 4213 records the stimulus content given at Step S101 in the storage unit 424, in association with the time information (Step S102).

As described above, the third information processing apparatus 42 of the present embodiment records the time information transmitted from the time management apparatus 10 in association with the stimulus content of the stimulus that is given to the subject P. Therefore, the biological information measurement system 2 is able to obtain the brain function data, the head position data, and the chronological data of the the stimulus content that can be temporally synchronized with one another without imposing a burden on the subject P, so that it is possible to measure the brain function with high accuracy.

Meanwhile, the embodiment as described above may be embodied with various modifications by changing a part of the configurations and functions of each of the apparatuses as described above. Therefore, in the following, some modifications of the embodiment as described above will be described as other examples. In the following, a difference from the embodiment as described above will be mainly described and detailed explanation of components common to those as described above will be omitted. Further, the modifications described below may be implemented individually or may be implemented by an appropriate combination.

First Modification

In the present modification, a configuration in which, with use of the information provided by the providing apparatus 41, transmission of the time information between the time management apparatus 10 and the brain function measurement apparatus 20 or between the time management apparatus 10 and the head position measurement apparatus 30 is not needed will be described. It is assumed that the providing apparatus 41 is a certain apparatus, such as a projector, that projects an output video on a screen. The stimulus instruction unit 4212 of the third information processing apparatus 42 according to the present modification instructs the providing apparatus 41 to output certain information that provides the time information transmitted from the time management apparatus 10 and a timing based on the time information, in such a manner that the information is included in a video to be output.

Specifically, the stimulus instruction unit 4212 displays, in a part of an output video of the providing apparatus 41, information (hereinafter, also referred to as a marker image) for providing the time information or a timing based on the time information. Here, the marker image may be, for example, blinking of light or in a form of a time code. Further, it is preferable that the marker image is displayed at a position at which a video for a visual stimulus is not disturbed and has a certain size that does not change a natural reaction to a visual stimulus. Meanwhile, the marker image may be continuously displayed or may be displayed at a predetermined timing (for example, a period in which a stimulus is provided, or the like).

In contrast, the first information processing apparatus 22 or the second information processing apparatus 32 is connected to a sensor apparatus that is arranged so as to be able to detect the marker image displayed on the screen. As the sensor apparatus, for example, an optical detector, such as a photodiode, an imaging device, such as a charge coupled device (CCD) or a complementary metal oxide semiconductor (CMOS), or the like may be used.

Then, the time information acquisition unit 2211 of the first information processing apparatus 22 or the time information acquisition unit 3211 of the second information processing apparatus 32 acquires the time information on the basis of the marker image detected by the sensor apparatus. With this configuration, the first information processing apparatus 22 or the second information processing apparatus 32 is able to temporally synchronize the brain function data and the head position data on the basis of the time information measured by the time management apparatus 10, without receiving the time information from the time management apparatus 10 similarly to the embodiment as described above. Therefore, it is possible to achieve the same effects as described above.

Furthermore, in the configuration of the present modification, the time management apparatus 10 and the first information processing apparatus 22 need not perform electrical communication with each other, so that it is possible to reduce an electromagnetic interference caused by measurement of the brain function of the subject P.

Meanwhile, if the configuration of the present modification is applied to the second information processing apparatus 32, the image capturing apparatuses 31 may be used as the sensor apparatus that detects the marker image. In this case, the image capturing apparatuses 31 are arranged at positions at which it is possible to capture images of a part of the face or the head of the subject P and capture the marker image to be displayed on the screen. Further, the time information acquisition unit 3211 of the second information processing apparatus 32 acquires the time information from the marker image included in the captured image data.

Second Modification

In the embodiment as described above, the configuration in which the time management apparatus 10 is an independent apparatus has been described, but embodiments are not limited to this example, and a different apparatus, such as the third information processing apparatus 42 may have the functions of the time management apparatus 10.

For example, if the third information processing apparatus 42 has the functions of the time management apparatus 10, the third information processing apparatus 42 transmits time information that the third information processing apparatus 42 has measured to the first information processing apparatus 22 and the second information processing apparatus 32. The third information processing apparatus 42 also records the stimulus content that is provided by the providing apparatus 41, in association with the time information that the third information processing apparatus 42 has measured.

With this configuration, in the configuration according to the present modification, similarly to the embodiment as described above, it is possible to synchronize the brain function data, the head position data, and the stimulus content. Therefore, it is possible to achieve the same effects as those of the embodiment as described above.

Third Modification

In the embodiment as described above, the mode in which the stimulus providing apparatus 40 operates independently of the other apparatuses has been described, but embodiments are not limited to this configuration. For example, the stimulus providing apparatus 40 may control the stimulus content provided to the subject P and the providing timing in accordance with a measurement result or the like of the head position measured by the head position measurement apparatus 30. A configuration according to the present modification will be described below.

In the present modification, for example, the second information processing apparatus 32 and the third information processing apparatus 42 are connected to each other, and the head position data measured (estimated) by the second information processing apparatus 32 is transmitted to the third information processing apparatus 42. Further, the third information processing apparatus 42 receives the head position data transmitted from the second information processing apparatus 32, and stores the head position data in the storage unit 424 in association with the time information transmitted from the time management apparatus 10. Meanwhile, the third information processing apparatus 42 may have the functions of the head position data recording unit 3214 and the synchronization processing unit 2214.

Furthermore, the stimulus instruction unit 4212 of the third information processing apparatus 42 is able to selectively give instructions on a plurality of kinds of visual stimuli. Moreover, the stimulus instruction unit 4212 of the third information processing apparatus 42 analyzes a movement amount or the like of the subject P from the head position data, and changes a condition for providing the visual stimuli in accordance with an analysis result.

An example of operation performed by the third information processing apparatus 42 will be described below.

First, an example will be described in which any of two kinds of visual stimuli A and B (a stimulus period for each of the stimuli is 3 seconds) is provided 50 times in random order. Hereinafter, operation of providing the visual stimuli will also be referred to as a “trial”.

When the trial as described above is performed, for example, the brain function data that is measured for each of the visual stimuli is averaged among trials, and an activity intensity, a peak latency, or the like of a specific brain area in a specific time period is examined, so that a state of the specific brain area can be examined.

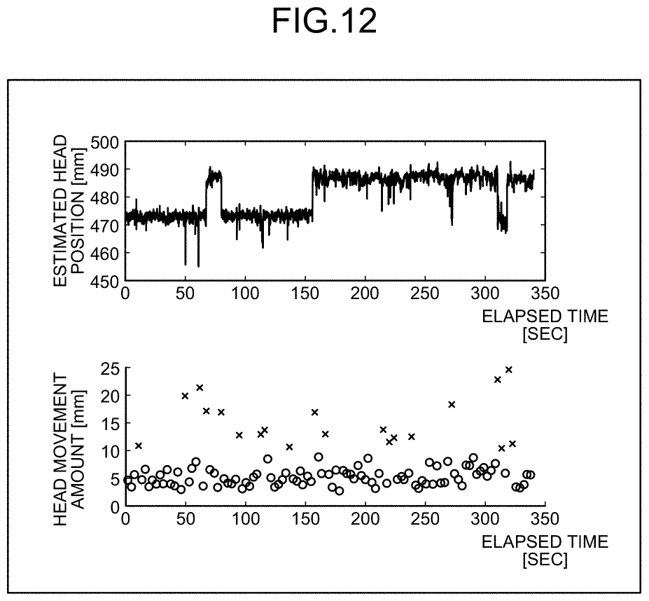

FIG. 12 is a diagram illustrating an example of a temporal change in the head position according to the present modification. In FIG. 12, an upper graph is a diagram illustrating a temporal change in the head position of the subject P that is estimated by the second information processing apparatus 32. Further, a lower graph is a diagram illustrating a temporal change in a movement amount that is derived from the upper graph. In each of the graphs, a horizontal axis represents an elapsed time, and a vertical axis represents an amount of change in the position. Here, a head movement amount may be derived by the second information processing apparatus 32 or the third information processing apparatus 42.

For example, the head movement amount may be derived from a difference between temporally consecutive head positions at a predetermined time interval. Meanwhile, in FIG. 12, plots at which the movement amount is smaller than 10 mm are represented by white circles and plots at which the movement amount is equal to or larger than 10 mm are represented by cross marks.

In this case, the stimulus instruction unit 4212 performs a trial for providing any of the visual stimuli A and B at an interval of 3 seconds, and calculates the movement amount from a difference between the head positions before and after the trial on the basis of the head position data measured by the second information processing apparatus 32. Further, the stimulus instruction unit 4212 may count, on the basis of a threshold for the movement amount, that is, a threshold for an amount of change in the posture, that is determined in advance, the number of times the movement amount exceeds the threshold and may control the number of times to give the visual stimuli in accordance with the counted number. For example, if the movement amount of “10 millimeters (mm)” is set as the threshold, the stimulus instruction unit 4212 counts the number of times the movement amount is smaller than 10 mm or the movement amount is equal to or larger than 10 mm, for each of visual stimulus conditions.

As one example, it is assumed that a measurement condition (hereinafter, also referred to as the number of trials) is set such that a state in which the movement amount is smaller 10 mm occurs 50 times or more for each of the visual stimuli A and B. In this case, the stimulus instruction unit 4212 counts the number of times the head moves by 10 mm or more when a predetermined time elapsed. Here, for example, if it is assumed that the number of times the head moves by 10 mm or more is 16 in a total of 100 times when 300 seconds elapsed, the stimulus instruction unit 4212 determines that the condition for the number of trials is not met. In this case, the stimulus instruction unit 4212 additionally performs the trials a predetermined number of times (for example, 10 times) for each of the visual stimuli A and B.

Then, if the stimulus instruction unit 4212 confirms that the state in which the movement amount is smaller than 10 mm occurs 50 times or more for each of the visual stimuli A and B due to addition of the trials, the stimulus instruction unit 4212 determines that the number of trials is met and stops providing the stimuli to the subject P. Accordingly, the stimulus instruction unit 4212 is able to automatically perform the planned number of trials.

Meanwhile, the stimulus instruction unit 4212 may count the number of times of occurrence of the state in which the movement amount is smaller than 10 mm for each of the visual stimulus conditions while providing the stimuli to the subject P, and may automatically continue to provide the stimuli to the subject P until the planed number of trials is met.

Further, in the example as described above, the stimulus instruction unit 4212 is configured to automatically continue to provide the stimuli, but it may be possible to confirm with a user (measuring person) about whether to continue to provide the stimuli at a predetermined timing. In this case, the third information processing apparatus 42 includes the same display unit and the same operation unit as the display unit 225 and the operation unit 226 of the first information processing apparatus 22, and confirms with the user via the display unit and the operation unit. Specifically, the stimulus instruction unit 4212 causes the display unit to display a screen for confirming whether to continue to provide the stimuli, and receives, via the operation unit, operation of giving an instruction on whether to continue to provide the stimuli.

For example, the stimulus instruction unit 4212 may confirm whether to continue the providing if a time period during which the stimuli are being provided reaches a predetermined time, or may confirm whether to continue the providing if the number of times of trials reaches a predetermined number. Further, the stimulus instruction unit 4212 may confirm whether to continue the providing if a ratio of the number of times the head moves by 10 mm or more to the number of trials reaches a predetermined value or more (for example, 50% or more).

Furthermore, the stimulus instruction unit 4212 may be configured to receive the number of trials to be performed from the user. In this case, the stimulus instruction unit 4212 performs the trials the same number of times as instructed by the user, and confirms again whether to continue providing if the number of trials does not reach the scheduled number. Meanwhile, it is preferable that, when confirming with the user about whether to continue to provide the stimuli, the stimulus instruction unit 4212 simultaneously displays the number of trials for each of the visual stimulus content, statistical information on the movement amount, or the like.

As described above, by receiving input from the user as to whether it is necessary to provide a stimulus, it is possible to continue to provide the stimulus only when needed, so that it is possible to improve usability and reduce a burden on the subject P.

Meanwhile, a program executed by each of the apparatuses in the first embodiment, the second embodiment, and the modifications as described above is provided by being incorporated in a ROM, a storage unit, or the like. The program executed by each of the apparatuses in the first embodiment, the second embodiment, and the modifications as described above may be provided by being recorded in a computer readable recording medium, such as a compact disk-ROM (CD-ROM), a flexible disk (FD), a compact disk-recordable (CD-R), or a digital versatile disk (DVD), in a computer-installable or computer-executable file format.

Furthermore, the program executed by each of the apparatuses in the first embodiment, the second embodiment, and the modifications as described above may be stored in a computer connected to a network, such as the Internet, and provided by download via the network. Moreover, the program executed by each of the apparatuses in the first embodiment, the second embodiment, and the modifications as described above may be provided or distributed via a network, such as the Internet.

According to an embodiment, it is possible to measure a brain function with high accuracy without imposing a burden on a subject.

The above-described embodiments are illustrative and do not limit the present invention. Thus, numerous additional modifications and variations are possible in light of the above teachings. For example, at least one element of different illustrative and exemplary embodiments herein may be combined with each other or substituted for each other within the scope of this disclosure and appended claims. Further, features of components of the embodiments, such as the number, the position, and the shape are not limited the embodiments and thus may be preferably set. It is therefore to be understood that within the scope of the appended claims, the disclosure of the present invention may be practiced otherwise than as specifically described herein.

The method steps, processes, or operations described herein are not to be construed as necessarily requiring their performance in the particular order discussed or illustrated, unless specifically identified as an order of performance or clearly identified through the context. It is also to be understood that additional or alternative steps may be employed.

Further, any of the above-described apparatus, devices or units can be implemented as a hardware apparatus, such as a special-purpose circuit or device, or as a hardware/software combination, such as a processor executing a software program.

Further, as described above, any one of the above-described and other methods of the present invention may be embodied in the form of a computer program stored in any kind of storage medium. Examples of storage mediums include, but are not limited to, flexible disk, hard disk, optical discs, magneto-optical discs, magnetic tapes, nonvolatile memory, semiconductor memory, read-only-memory (ROM), etc.

Alternatively, any one of the above-described and other methods of the present invention may be implemented by an application specific integrated circuit (ASIC), a digital signal processor (DSP) or a field programmable gate array (FPGA), prepared by interconnecting an appropriate network of conventional component circuits or by a combination thereof with one or more conventional general purpose microprocessors or signal processors programmed accordingly.

Each of the functions of the described embodiments may be implemented by one or more processing circuits or circuitry. Processing circuitry includes a programmed processor, as a processor includes circuitry. A processing circuit also includes devices such as an application specific integrated circuit (ASIC), digital signal processor (DSP), field programmable gate array (FPGA) and conventional circuit components arranged to perform the recited functions.

What is claimed is:

1. A biological information measurement system comprising:
a time measurement apparatus configured to transmit time information;
a first measurement apparatus configured to measure brain neural activity of a subject, based on a biological signal detected from the subject;
a first recording apparatus configured to record first data indicating a temporal change in the brain neural activity measured by the first measurement apparatus, in association with the time information received from the time measurement apparatus;
an image capturing apparatus configured to capture an image of the subject; and
a second recording apparatus configured to record second data indicating a temporal change in a posture of the subject, the posture identified based on the image captured by the image capturing apparatus, in association with the time information received from the time measurement apparatus.

2. The biological information measurement system according to claim 1, wherein the second recording apparatus is configured to identify, as the posture of the subject, at least one of a position, an orientation, and a movement amount of a face of the subject.

3. The biological information measurement system according to claim 1, wherein the time measurement apparatus is implemented by one of the first recording apparatus and the second recording apparatus.

4. The biological information measurement system according to claim 1, wherein
the first recording apparatus further includes a first time measurement unit configured to start to measure time in synchronization with the time information received from the time measurement apparatus, and
the first recording apparatus is configured to record the first data in association with time information for which time measurement is performed by the first time measurement unit.

5. The biological information measurement system according to claim 1, wherein
the second recording apparatus further includes a second time measurement unit configured to start to measure time in synchronization with the time information received from the time measurement apparatus, and
the second recording apparatus is configured to record the second data in synchronization with time information for which time measurement is performed by the second time measurement unit.

6. The biological information measurement system according to claim 1, further comprising:
a providing apparatus configured to provide a stimulus to the subject; and
a third recording apparatus configured to control stimulus content provided by the providing apparatus, and records the stimulus content provided by the providing apparatus, in association with the time information received from the time measurement apparatus.

7. The biological information measurement system according to claim 6, wherein the third recording apparatus is configured to change a condition related to providing the stimulus content, based on the second data.

8. The biological information measurement system according to claim 7, wherein the third recording apparatus is configured to count a number of times an amount of change in the posture of the subject exceeds a threshold, based on the temporal change in the posture represented by the second data, and control a number of times providing the stimulus content, based on the counted number.

9. The biological information measurement system according to claim 6, wherein the third recording apparatus is configured to receive, from a user, a number of times providing the stimulus content, and causes the providing apparatus to provide the stimulus content the received number of times.

10. The biological information measurement system according to claim 6, wherein the time measurement apparatus is implemented by the third recording apparatus.

11. The biological information measurement system according to claim 1, wherein the second data comprises a representative value in a predetermined time.

12. The biological information measurement system according to claim 1, wherein measurement of the brain neural activity comprises electrophysiological measurement.
